(19) United States
(12) Patent Application Publication
(10) Pub. No.: US 2006/0127518 A1
(43) Pub. Date: Jun. 15, 2006

(54) Red nocardia cell wall skeleton preparation process and its therapeutic use on treating cervical erosion

(75) Inventors: Ce Zhang, Shenyang (CN); Yi Zhang, Shenyang (CN); Guoying Zhang, Shenyang (CN); Xiaoming Hong, Shenyang (CN)

Correspondence Address: MERCHANT & GOULD PC, P.O. BOX 2903, MINNEAPOLIS, MN, 55402-0903, US

(73) Assignee: Shenyang Sunbellcom Bio-Pharmaceutical Co., Shenyang (CN)

(21) Appl. No.: 10/525,849

(22) PCT Filed: Sep. 5, 2003
(86) PCT No.: PCT/CN03/00751

§ 371 Date: Oct. 24, 2005

(51) Int. Cl. A61K 35/74

(52) U.S. Cl. 424/780

(57) ABSTRACT

This invention relates to a biological preparation, especially Nocardia Rubra cell wall skeleton preparation. After cultivating, collecting thallus, cell fragmentation, purifying enzyme, removing the grease, the final preparation containing Nocardia Rubra cell wall skeleton is obtained by dispensing, lyophilizing and filling. The invented preparation has marked effect to adaptable diseases, healing rate is 88%, total validity rate is 100%. This external preparation shows harmless, safe, non-toxicity and easy to be used with little side effect and lower cost.

### TECHNICAL FIELD

The present invention is related to a kind of microbial preparation produced by bioengineering technology, which is a preparation of Nocardia Rubra cell wall skeleton. The main component of the preparation is Nocardia Rubra cell wall skeleton that contains Arabic semi-glactan, muramic acid and mucopeptide etc. Moreover, the invention is also related to the process and detective method of the preparation, as well as its effect on the treatment of cervical erosion.

### BACKGROUND OF THE INVENTION

Cervical erosion is the most common chronic disease in gynecology, and about 30 percent distaff of pregnant age are suffering from it. The symptom may be increased leucorrhea, lumbosacral pain and bearing down. If the local is inflammatory, pyogenic leucorrhoea will appear, and therapy must be carried now. As long as severe cervical erosion is not dealt with in time, it will easily lead to canceration.

At present, the physical therapy generally used includes electrotherapy, freezing, laser and microwave etc. But the methods mentioned above will bring the patient variety of side effect, such as injury of normal tissue, excessive vaginal secretion accompanied by bad smell. Simultaneously, the effect is not satisfactory and also constrained by season. It is not suitable to apply them for therapy in summer.

Nowadays, the medicine we frequently use in dealing with the patient includes embrocation, embolism, effervesce and the like. But medicines have many shortcomings, e.g. un-effectiveness, high relapse probability, a lot of side effects and even tend to break the microbial ecological balance of internal environment in vagina.

In a word, ideal therapy has not been found out to deal with cervical erosion now. Most patients have no choice but to endure the pain caused by these therapies.

### SUMMARY OF INVENTION

In order to overcome the shortcoming of the prior arts, the object of the present invention is to provide a kind of Nocardia Rubra cell wall skeleton preparation and its preparation method.

Another object of this invention is to apply the preparation containing Nocardia Rubra cell wall skeleton in the treatment of cervical erosion.

The invented preparation has distinct effects on indication, less side reaction, no obvious local irritating symptom and no injury to normal tissue. At the same time, it can lead to deceased secretion, improved property and warranted safety. It is for convenient use with low cost.

To carry out the objects, the invention provides a kind of preparation to deal with cervical erosion, characterized by containing Nocardia Rubra cell wall skeleton.

The preparation can also contain the pharmaceutically acceptable carriers. The Nocardia Rubra had been deposited in China General Microbiological Culture Collection Center in Feb. 5, 2002 with deposition No.: CGMCC. No. 0712

The pharmaceutically acceptable carriers comprise saccharide (monosaccharide, disaccharide, polysaccharide), glycoride (e.g. Dextran), fat (fatty acid included), protein( e.g. albumin, gelatin), amino acid (e.g. sodium Glutamate, glycine, cysteine) and the like, and lipid (e.g. polysorbate), alcohol (glycerine, propylene glycol, mannitol), dimethyl sulfoxide organic solvent, hydroxyl-carboxy acid (e.g. sodium citrate), polyanion (e.g. Polyphosphate), antisepsis, antioxidation etc, but dextran is preferable.

Based on the total weight of the preparation, about 0.005-99 weight percent is Nocardia Rubra cell wall skeleton.

The invention also provides with a method of the preparation, comprising: Obtaining product of cell wall skeleton after culture, collection, cell shattering, enzyme purification, delipidation of Nocardia Rubra with deposit No. CGMCC. No. 0712;

Mixing the production of Nocardia Rubra cell wall skeleton and the pharmaceutically acceptable carriers to obtain a preparation.

During the course of preparation, dextran is preferably pharmaceutically acceptable carrier. Based on the total weight of the preparation, Nocardia Rubra cell wall skeleton is in amount of about 0.005-99 weight percent.

The present invention also provides a treatment method of cervical erosion, characterized by applying pharmaceutically effective amount of Nocardia Rubra cell wall skeleton preparation directly on a suffering site of a subject.

### Microbe Required in the Invention

The bacteria strain used to produce the preparation of in the invention belongs to Nocardia Rubra which possess the ability to produce Nocardia Rubra cell wall skeleton preparation. Nocardia Rubra Nr-8206 has the above properties and is suitable to produce the preparation. After cultured 5 days at 33C on glycerine agar media, the bacteria will be obtained, and it had been deposited in China General Microbiological Culture Collection Center in Feb. 5, 2002, deposit No. CGMCC. No. 0712.

### Bacterial Property of the Bacteria in the Invention

1. Culture Property

The bacterium, Nocardia Rubra is inoculated on glycerine agar media of pH 7.2˜7.5, and cultured for 48 hours at 33° C., the bacterial colony will be eminent and appear orange, and dry with granular appearance, and a bit luster can be seen. Inoculation ring is vulnerable to touch, and mycelial body formation cannot be seen under the laboratory condition.

2. Morphological Property of Bacterium

The bacterium has positive Gram-staining and negative anti-acid staining. The bacterium is branch shaped with transverse membrane, forming thin mycelia body. The whole mycelia split to irregular column shaped short thick cell, after cultured for five days, it will appear short rod-like and spherical.

3. Biochemical Reaction

The bacterium shows fermentation of mannitol, sorbate; and no fermentation of lactose, maltose, sucrose, synanthrin, rhamnose, arabinose, muscarinose, gossypose, semi-lactose, amylum gelatin as well as positive of nitrate reduction.

Production would be made if above criteria could be met with culture and extract method.

By the way of microbial production, the bacteria in the invention with Nocadia Rubra genus can be used. After secondary culture, amplification, ultrasound waves shattering, cell wall skeleton is extracted, followed by enzyme purification, delipidation. Adding appropriate amount of excipient, then the culture will be made by freezing to dry. Both solid and liquid culture can be applied in the present invention.

There is no special requirement for nutritional source in culture media, it may contain carbon, nitrogen source and other nutritional sources that generally used in microbial culture. Carbonic sources can be such as amylum, amylin, mannital, sucrose, lactose, sorbate, maltose, and etc. Nitrogen sources can be meat extract, peptone, ammonium, nitrate and other organic or inorganic nitrogenic compound. Some inorganic salt to other source of nutrition, e.g. phosphate can be included also optionally.

There's no strict requirement for culture condition, e.g. time and temperature whenever culture condition that conductive to the growth of bacteria and high output is used. For instance, pH level should fluctuate around neutralization, culture temperature being about 22˜37° C. Of course, the components, concentration of hydrogen ion, culture temperature should be adjusted according to different bacteria and external condition etc to obtain the best outcome.

By the way of microbial culture, the bacteria in the invention are inoculated on glycerine agar media to culture. After further culture of qualified bacteria, collection, cell shattering, enzyme purification and delipidation, the active component cell wall skeleton can be obtained. Then pharmaceutically acceptable carrier known by the person skilled in the art, e.g. acceptable excipient, preferably such as dextran may be added, followed by freezing it to be become a commercial product after grouting.

The dosage of the product in this invention may be, but not limited, 0.5 ml per bottle.

Component and proportion may be as following:

| | | |
|---|---|---|
| Nocardia Rubra cell wall skeleton | 5-1000 | μg |
| Dextran | 0.01-100 | mg |

By detection of the bacteria strain, culture liquid, the index of the quality of finished product can be seen in the following tables.

Examination of bacterial strain:

| Content of test | Criteria of test | Result of test |
|---|---|---|
| Characteristics | The strain is swelling up, | The strain is swelling up, |
| of cultivation | yellow, with the surface | yellow, with the surface |
| | dry, wrinkle, sand granules- | dry, wrinkle, sand granules- |
| | like and luster. | like and luster. |
| | The inoculate ring is fragile | The inoculate ring is fragile |
| | and will not form the aerial | and will not form the aerial |
| | mycelium in the lab. | mycelium in the lab. |
| Morphologic | Gram-staining is positive, | Gram-staining is positive, |
| character of | and acid-fast staining is | and acid-fast staining is |
| thallus | negative. | negative. |
| | Thallus is branching and | Thallus is branching and |
| | having diaphragm, which is | having diaphragm, which is |
| | forming the fine mycelium. | forming the fine mycelium. |
| | The whole mycelium is | The whole mycelium is |
| | divided into irregular | divided into irregular |
| | short column shape cell, | short column shape cell, |
| | and will turn into short | and will turn into short |
| | stem shape and spherical | stem shape and spherical |
| | shape after 5 days culture. | shape after 5 days culture. |
| Biochemical | It can ferment manicol, | It can ferment manicol, |
| reactions | sorbitol; cannot ferment | sorbitol; cannot ferment |
| | lactose, maltose, sucrose, | lactose, maltose, sucrose, |
| | inulin, rhamnose, | inulin, rhamnose, |
| | arabinose, adonitoxolo- | arabinose, adonitoxolo- |
| | genin, mannose, gossupose, | genin, mannose, gossupose, |
| | galactose, starch, animal | galactose, starch, animal |
| | glue; nitrate reduction | glue; nitrate reduction |
| | examination is positive. | examination is positive. |

Examination of stock solution:

| Content of test | Criteria of test | Result of test |
|---|---|---|
| Determination of total amount of | 0.9%˜1.4% | 0.9%˜1.4% |
| solid | | |
| Determination of sugar content | ≧2.5 mg/ml | ≧2.5 mg/ml |
| Determination of muramic acid | ≧200 μg/ml | ≧200 μg/ml |
| Determination of protein remnant | ≦30% | ≦30% |
| Determination of lipid remnant | ≦5% | ≦5 |
| Determination of RNA and DNA | ≦5% respective | ≦5% respective |
| remnant | | |
| Determination of TritonX-100 | ≦5% | ≦5% |
| remnant | | |
| Aseptic test | Negative | Negative |

Examination of final product:

| Content of test | Criteria of test | Result of test |
|---|---|---|
| Physical Character | The production is white | White loose bodies |
| | loose bodies or power | |
| Moisture content | ≦6% | 2.74% |
| Solubility | Dissolve in 1 minute | Certified |
| | after the addition of 0.9% | |
| | sodium chloride solution. | |
| Sugar differential test | Solution is blue-green | Solution is blue-green |
| | color | color |
| Content of muramic | ≧1.0 μg/bottle | 1.64 μg/bottle |
| acid | | |
| Abnormal toxicity | Mice should be alive | Certified |
| experiment of mice | during the observation | |
| | with no abnormal | |
| | reaction, and the weight | |
| | of mice should increase | |
| | by the end of the | |
| | observation. | |
| Abnormal toxicity | Cavy should be alive | Certified |
| experiment of | during the observation | |
| cavy | with no abnormal | |
| | reaction, and the weight | |
| | of cavy should increase | |
| | by the end of the | |
| | observation | |
| Efficacy experiment | Phagocytosis percentage: | Phagocytosis |
| | ≧10% | percentage: 43.6% |
| | Phagocytosis index: | Phagocytosis index: |
| | ≧0.15 | 0.63 |
| Aseptic test | Negative | Negative |

| | |
|---|---|
| Conclusion of | Certified |
| examination | |

Compared with other available techniques, this invention is the biological preparation using microbial cell wall skeleton as its effective component, which is a kind of immunopotentiator. Thus it will have the anti-tumor function of organism, prevent the infection from some virus and bacteria, and have phagocytosis ability of macrophage. This is identified by a series of experiments and clinical trials. The treatment effect is satisfactory with the healing rate reached to 88%, general effective rate reached to 100%. And it has no injury to normal tissue, non-toxicity, safety and little side effects. It is also a kind of external preparation for cervical erosion with high efficiency, good safety, convenient and low cost.

Nocardia Rubra used in this invention was already deposited in China General Microbiological Culture Collection Center on February 5, 2002 with the Deposit serial number: CGMCC No. 0712. And the test showed that the preserved strains are alive with no inactivation.

1. General Pharmacological Test for the Invented Product:

1). The blood pressure, respiratory, heart rate and ECG of anesthetic cats had no obvious change after the cats were intravenous injected by the preparation whose dosage was 20, 40 and 80 times of clinical dose.

2). The coordination exercise and memory function of mice had no obvious change after the mice were intravenous injected by 0.5 ml preparation whose dosage was 1000 times of clinical dose.

So the preparation of this invention has shown that no obvious effect on the psychoneurous system, cardiovascular system and respiratory system of animal are found substantially.

2. Safety test: (Aseptic, Toxicity)

1). Result of aseptic test is negative; identifying the aseptic test is certified.

2). Acute toxicity test of mice:

The mice in trial group were subcutaneous or abdominal injected by the preparation whose dosage is five times of human. And the mice in contrast group were injected by the aseptic saline instead. After 7-8 days observation, the condition of mice was normal, their weight was increasing, and the necropsy had no abnormal findings. So the toxicity test was also certified.

3. Long Term Toxicity Test:

After three months of administration to this preparation by vagina (30 times of clinical dosage), the trial dogs remained normal conditions. No toxicity on dogs was found and the ECG, hemanalysis index of dogs remained normal, so did two weeks later, which showed that no delayed toxicity reaction existed.

4. Stability Test of the Preparation:

Under the same condition of technology and in normal temperature, compared with the quality on output date, the content of alanine and muramic acid in the preparation had no obvious difference after placing for 1, 2, 3, 8, 14 and 21 months. This shows the good stability of the preparation. And from the efficacy test, the phagocytosis percentage and index also has no obvious change. After lyophilized, the preparation can be preserved for two years in normal temperature.

5. Immunity Test:

The ability of surface immunophagocytosis of this preparation is very potential, with the phagocytosis percentage ≧10%, phagocytosis index ≧0.15. In fact the phagocytosis percentage and index of dextran are even lower than saline, this shows the potential immunophagocytosis ability of this preparation which dextran and saline have not.

### EXAMPLE

The glycerine-agar culture medium is formed by 2.0-6.0 g of beef extract, 4.0-10.0 g of peptone, 2.0-6.0 g of sodium chloride, 10.0-20.0 g of agar, 4.0-10.0 ml of glycerine, 0.1-0.5 g of Na₂HPO₄.12H₂O, and 500 ml-1000 ml of distilled water. After sterilization under pH 7.0-8.0, Nocardia Rubra Nr-8206, CGMCC No. 0712 is inoculated into this culture medium for two-eight days in 28-36° C., to have a culture, then after washing off the lawn by aseptic distilled water, the culture will be centrifuged at 1000-5000 rpm for 5-40 minutes, following by collecting bacterial and washing 1-7 times, weighing the wet weight and preserving it in −70° C.-20° C. After the purification test, the culture having no other mixed bacteria can be used. 1-5 portion wet bacteria is oscillated with 1-5 portion aseptic distilled water, and shattering them by ultrasound disintegrator to have a diluted culture. To take gram-staining and examination by microscope every 5-30 minutes, it will be certified at least 5 visual fields each has less than 15 tangible bacteria. Then such culture is got rid of the residual by 1000-2000 rpm centrifugation for 5-40min, and the supernatant is preserved in −70° C.-20° C. The phosphate PBS buffer solution in PH 7.0-8.0 is well prepared and preserved after sterilization. And then the cell wall skeleton is extracted. 100-500 ml supernatant is centrifuged at 10000-20000 rpm for 5-40 minutes, followed by excluding supernatant and mixing the sediment with PBS buffer solution which contains DNA and RNA enzyme of 100-500 μg/ml in 15-30° C. for 0.5-3 hours to get a mixture. Then the mixture is further centrifuged at 10000-20000 rpm for 5-40 minutes, and followed by washing the sediment 1-5 times by PBS buffer solution. The sediment is then tested by RNA and DNA enzyme digestive rate, diluted to 100-500 ml by 0.5-5% polyethylene glycol nonylphenyl ether for 12-48 hours in 15-30° C. It is centrifuged at 10000-20000 rpm centrifugation for 5-40 minutes and disposing a supernatant again to get the another sediment. Such sediment is washed by PBS buffer solution for 1-5 times. The sediment is diluted by the solution which contains pronase (50-200 μg/ml) and trypsin (1.0-5.0 mg/ml) to 100-500 ml for 8-24 hours in 18-30° C., and then continuously centrifuged at 10000-20000 rpm for 5-40 minutes to get a sediment again. The sediment is also washed by PBS buffer solution for 1-5 times. Accordingly 0.5 ml sample to test the protein remnant is kept well.

In the enzyme purification, the enzyme known by those skilled in the art can be used to cut the protein or peptide of the Nocardia Rubra cell wall into desired pieces for the preparation.

Get Rid of Lipid by Organic Solvent:

The sediment finally obtained as mentioned above is diluted by acetone to 100-500 ml for 12-48 hours in 18-30° C., and then centrifuged at 10000-20000 rpm for 5-40 minutes to get sediment, followed by washing by PBS buffer solution for 1-5 times. Then the sediment is diluted by the solution which contains diethylether and ethyl alcohol 1:0.5-1:5 to 100-500 ml for 12-18 hours in 18-30° C., and further centrifuged at 10000-20000 rpm for 5-40 minutes to get sediment again. Such sediment is also washed by PBS buffer solution for 1-5 times. The lipid remaining rate is tested.

After above steps, the sediment will be the cell wall skeleton. The sediment is weighed the wet weight and diluted with 10-100 mg/ml of aseptic distilled water. After sterilization, it is preserved in −70° C.-10° C.

The canned lyophilized procedure: (To dispense 5000 ml semi-manufactured goods for example)

The semi-product is composed of 50-1000 mg Nocardia Rubra cell skeleton, 0.1-1000 g dextran, aseptic injective water 5000 ml. Then the semi-product is mixed by magnetic blender. 0.5 ml to every bottle is canned and then lyophilized well. The finished product is tested to ensure that every bottle contains 5-1000 μg Nocardia Rubra cell wall skeleton, 0.01-100 mg dextran. The product can be preserved 2 years in normal temperature.

Clinical Trial:

The clinical trial of this preparation has been taken in the Woman and Children hospital in Shenyang, China and the results were given as follows:

A. Research Method: Opened and Multiple-Central Randomized Contrast Study.

A-A. Requirements of Tested Objects:
1. Age was between 23 and 45, married and non-pregnant woman.
2. Cervical erosion, excluding the cancerization ones.
3. Menstruation was regular and menstrual cycle was not shorter than 25 days.
4. able to persist to finish the therapeutic course.
5. Not take other therapies during tested phase.

A-B. Exclusion Requirements of Tested Objects:
1. The result of vaginal cellular smear-inspection was over II_{B}.
2. The cervix was cancerization.
3. With various vaginal inflammations or acute, sub-acute pelvic inflammatory diseases.
4. During postpartum 3-months.
5. With allergic history to many drugs.

A-C. The Number of Tested Objects:
75 cases in all, and were distributed according to 2:1 randomized table: 50 cases in trial group, and 25 cases in contrast group.

A-D. Drugs Used and Usage:

1. Trial Group:

The drug was an preparation of Nocardia Rubra cell wall skeleton. It could be made into embrocation. The norm was that every bottle had 60 μg frozen-dry-powder of Nocardia Rubra cell wall skeleton (short for Nr-CWS), which was the invention of applicant's company. The batch number was 950321.

Usage: After eliminating the excretion on the cervical surface, the 60 μg frozen-dry-powder of the embrocation related above was dissolved in 2.0 ml physical saline, the aseptic bandage and hygienic cotton balls which have been soaked by the drug was put on the cervical erosion area. The patient took them out 24-hours later. The application was repeated as above twice every week, and 6 times in all.

2. Contrast Group:

The therapeutic drug was a kind of ALBOTHYL concentrate. There was 360 mg ALBOTHYL in 1 g of drug, which was a product of BYK Gulden D-78467 Konstanz, Germany. The batch number was: J940475.

Usage: Firstly, 1:5 dilution-fluid was used to wash the cervix, and the gauze-clot was used to eliminate the cervical mucus and intra-vaginal excretion. Then stick cottons were soaked with concentration-fluid to the erosion areas closely. The patient would take them out 3 minutes later. At this time, the erosion areas had all turned white. Application was repeated as above 2 times a week, and 6 times in all.

The tested objects of trial group and CONTRAST group all had the therapy 2-3 days after the menstruation was over.

A-E. The Diagnostic Criterion

1. Grading:
Mild degree: Erosion area is smaller than ⅓ of all cervical area.
Moderate degree: Erosion area is account for ⅓ to ⅔ of all cervical area.
Severe degree: Erosion area is over ⅔ of all cervical area.

2. Classification: According to the Depth Degree of Erosion:
Simple type: The surface of erosion is smooth.
Granular type: The surface of erosion is protruding in papilla form, not flat.

A-F. Observing Items:
1. Before using drugs:
   2) Inquired the history and did gynecological examination conventionally.
   3) Examined the clean-degree of vaginal secretion, which was classified into I, II, III degree. And examined Trichomonas and mold.
   4) Carried on cellular-smear examination to exclude cancerization.
   5) Measured the cervical erosion area by eye. Grading and classify the erosion according to diagnostic criterion.
   6) Did blood and urine routine tests. Examined hepatic function (GPT or ALT) and renal function (BUN, Cr).
   6) Arranged special people to proceed the therapy and observation in order to unify the criterion.
2. During the phase of using drugs:
   1) Suggested patients not do sexual intercourse during therapeutic phase.
   2) Inquired systemic or local adverse reactions of the drug.
   3 ) Observed local adverse reactions of vulva and vagina and change of leucorrhoea (character and amount) attentively every time before using drugs.
3. Post-Therapy:

The patients were reexamined after the next menstruation and continued to be asked symptoms and to observe local changes of cervical erosion (including area and depth) and the changes of amount and character of vaginal excretion. The therapeutic effect was evaluated. After the last time of using drug, the vaginal cellular examination and laboratory examination were repeated (blood and urine routine tests, hepatic and renal function tests).

A-G The Criterion of the Therapeutic-Effect-Judgment:

Cure: Erosion areas disappear, cervix is smooth, and symptoms disappear.

Effective: The erosion areas of superior and inferior labium contracts by 2 mm, and become more superficial. Symptoms are lightened.

No effect: Erosion areas are larger and deepen. Symptoms are severe.

A-H. Data Management and Statistical Methods:

United programs, united tables and united data managements as well as statistical method: X² test were done.

Results:

1 Comparison of the basic conditions

The patients with cervical erosion in the outpatient department are randomly divided into two groups: 50 cases in trial group curing with the preparation (embrocation) and 25 cases in the contrast group using ALBOTHYL. The basic conditions of the two groups are as the following table.

TABLE 1
Comparison of the selective patients in age, menarche age, pregnancy and delivery times

| | | | |
|---|---|---|---|
| Age | Menarche | Pregnancy times | Delivery times |

| | Cases | Mean | SD | Mean | SD | Mean | SD | Mean | SD |
|---|---|---|---|---|---|---|---|---|---|
| Trial group | 50 | 35.88 | 6.88 | 14.14 | 0.95 | 1.24 | 0.48 | 0.94 | 0.24 |
| Contrast group | 25 | 34.44 | 5.87 | 14.16 | 0.94 | 1.16 | 0.37 | 1.00 | — |

| | | | | |
|---|---|---|---|---|
| Significance of the two | P = 0.3737 | P = 0.9315 | P = 0.4349 | P = 0.2205 |
| groups' variance | | | | |

In the two groups, the youngest is 24 years old, and the oldest is 45 years old. The basic conditions have no obvious significant variance. That is to say, they are comparable.

2. Clinical Manifestation

2.1 Conditions of the Two Patient Groups in Degree and Types of Cervical Erosion

TABLE 2
Comparison of the two patient groups in degree and
types of cervical erosion

| | Pre-medication | | | | | | |
|---|---|---|---|---|---|---|---|
| | | mod- | | | The third re-examination | | |
| | mild | erate | severe | normal | mild | moderate | severe |
| Trial | 21 | 24 | 5 | 44 | 6 | — | — |
| group | | | | | | | |
| Contrast | 8 | 10 | 7 | 19 | 5 | 1 | — |
| group | | | | | | | |

Significance test of the trial group before medication and at the third re-examination (exact probability) shows P<0.01, so does the contrast group (P<0.01).

As showed in table 2 the mild erosions take 42%, the moderate take 48%, the severe take 10% in the trial group. While in the contrast group their percentages are separately 32%, 40% and 24%. After treatment, the normal gets to 88%, the mild reaches 12%. Meanwhile in the contrast group, the normal takes 76%, the mild takes 20%, the moderate takes 4%, which obviously shows that the trial group is prior to the contrast one.

TABLE 3
Comparison of the types of cervical erosion

| | Pre-medication | | | | The third re-examination | | |
|---|---|---|---|---|---|---|---|
| | Simple | granular | papillary | normal | Simple | granular | papillary |
| Trial group | 30 | 17 | 3 | 44 | 2 | 3 | 1 |
| Contrast group | 10 | 11 | 4 | 19 | 2 | 2 | 2 |

Note: In the tables, the normal refers to the filling of 0 in designing treatment effects. Significance test of pre-medication and the third re-examination in the trial group (exact probability) shows P<0.01.

From table 3, it can be found that in the pre-medication trial group simple types takes 60%, granular types take 34% and papillary types take 6%. In pre-medication contrast group, the three types show separately 40%, 44% and 16%. In post-therapy trial group, they separately take 4%, 6%, 2% and the normal takes 88%. In post-therapy contrast group, simple types take 4%, granular types take 4%, papillary types take 8% and the normal takes 76%.

TABLE 4
Change of leucorrhoea quantity and characteristics before and after medication

| | | | | first | | | secondary | | | third | | |
|---|---|---|---|---|---|---|---|---|---|---|---|---|
| | Pre-medication | | | re-exam | | | re-exam | | | re-exam | | |
| | Normal | more | much | Normal | more | much | Normal | more | much | Normal | more | much |
| Trial group | 27 | 21 | 2 | 45 | 4 | 1 | 50 | — | — | 50 | — | — |
| Contrast group | 13 | 7 | 5 | 18 | 6 | 1 | 21 | 4 | — | 23 | 2 | — |

Significance test of the trial group after medication and at the third re-examination (exact probability) shows P<0.0 1. So does the contrast group(P<0.0 1).

From the above table, conclusions can be made that

(1) In the trial group, before therapy the patients with normal leucorrhoea quantity take 54%, the patients with a little more leucorrhoea take 42%, the patients with much leucorrhoea take 4%. Meanwhile, in the contrast group, they take separately 52%, 28%, and 20%.

(2) After therapy, the patients with normal leucorrhoea quantity take 100%. In the contrast group the patients with normal leucorrhoea quantity take 92%, with a little more leucorrhoea take 8%. There is obvious variance both in the trial and contrast group (P<0.01).

From the percentage of the patients in these two groups, the normal takes 90% in the trial group, whereas in the contrast group they take separately 72% and 82%.

TABLE 5
Comparison of leucorrhoea characteristics

| | | | First | | Secondary | | Third | |
|---|---|---|---|---|---|---|---|---|
| | Pre-medication | | re-examination | | re-examination | | re-examination | |
| | Normal | Purulent | Normal | Purulent | Normal | Purulent | Normal | Purulent |
| Trial group | 44 | 6 | 48 | 2 | 49 | 1 | 49 | 1 |
| Contrast group | 18 | 7 | 23 | 2 | 25 | 1 | 25— | |

Significance test of the trial group after medication and. at the third re-examination (exact probability) shows P<0.01. So does the contrast group (P<0.01).

Table 5 shows that (A) the patients with normal leucorrhoea in the trial group before therapy take 88%, with purulent leucorrhoea take 12%. The patients with normal leucorrhoea in the contrast group before therapy take 72%, with purulent leucorrhoea take 24%. After therapy, the patients with normal leucorrhoea in the trial group take 98%, with purulent leucorrhoea take 2%, and the patients with normal leucorrhoea in the contrast group take 100%. After therapy, the patients with normal leucorrhoea in the trial group take 98%, and in the contrast group they take 100%. So conclusions can be made that the significance variance exists between the two groups both before and after therapy, but does not exist between the trial and contrast group (P>0.05), which may be due to the patients chief complaint of leucorrhoea quantity and characteristics.

2.3 Vaginal Clearance Degree, Cervical Smear and Papanicolaou Degree is Shown in Table 6.

TABLE 6
Vaginal clearance degree, cervical smear
and papanicolaou degree

| | |
|---|---|
| Vaginal clearance degree | Papanicolaou degree |

| | | | |
|---|---|---|---|
| Pre- | third re- | Pre- | third re- |
| medication | examination | medication | examination |

| | I | II | III | I | II | III | I | II | III | I | II | III |
|---|---|---|---|---|---|---|---|---|---|---|---|---|
| trial group | 22 | — | 28 | 47 | 3 | — | 30 | — | 20 | 49 | 1 | — |
| contrast group | 14 | — | 11 | 23 | 2 | — | 13 | — | 12 | 25 | — | — |

By statistics, the P value in the two groups before and after therapy are both less than 0.01 in vaginal clearance degree and papanicolaou degree by smearing. The obvious variance indicates the integrity and utility value of the preparation.

3. Therapy evaluation shown in table 7

TABLE 7
Therapy evaluation

| | Healed | effective | useless |
|---|---|---|---|
| Trial group | 44 | 6 | 0 |
| Contrast group | 19 | 6 | 0 |

In table 7, the healed rate in trial group is 88%, effective rate is 12%, and common effective rate is 100%. Moreover in the contrast group they are separately 72%, 28% and 100%. By x² test the variance is not obvious.

4.Safety and Side Effects

TABLE 8
side effects

| | Common cases | Yes | No |
|---|---|---|---|
| Trial group | 50 | 0 | 50 |
| Contrast group | 25 | 0 | 25 |

There is even no side effect both in the fifty cases of trial group and the twenty-five cases of contrast group, which indicates that the preparation (embrocation) is safe and reliable in the clinic.

TABLE 9
The results of laboratory test before and after therapy

| | | Bb (g/L) | RBC (10/L) | WBC (10/L) | BUN (mg/D1) | Cr (mg/D1) |
|---|---|---|---|---|---|---|
| | Cases | Mean ± SD | Mean ± SD | Mean ± SD | Mean ± SD | Mean ± SD |
| Trial group | | | | | | |
| pre-medication | 50 | 133.80 ± 9.02 | 3.95 ± 0.23 | 7.03 ± 1.95 | 4.45 ± 2.22 | 60.07 ± 18.49 |
| post-medication | 50 | 137.56 ± 3.69 | 3.96 ± 0.46 | 7.29 ± 1.23 | 4.20 ± 1.55 | 65.85 ± 21.88 |
| Difference of P value | | P = 0.0014 | P = 0.0806 | P = 0.1104 | P = 0.5527 | P = 0.1013 |
| before and after therapy | | | | | | |
| Contrast group | | | | | | |
| pre-medication | 25 | 137.76 ± 3.46 | 4.00 ± 0.15 | 6.80 ± 1.38 | 4.25 ± 1.26 | 74.82 ± 23.80 |
| post-medication | 25 | 138.52 ± 4.45 | 4.03 ± 0.13 | 7.20 ± 1.24 | 3.88 ± 1.37 | 68.23 ± 24.48 |
| Difference of P value | | P = 0.1119 | P = 0.4301 | P = 0.3115 | P = 0.3791 | P = 0.3205 |
| before and after therapy | | | | | | |

TABLE 10
Significance test of indexes in the trial and contrast
group before and after therapy

| | Rb | RBC | WBC | BUN | Cr |
|---|---|---|---|---|---|
| Before | P = 0.0704 | P = 0.3963 | P = 0.6048 | P = 0.8684 | P = 0.007 |
| therapy | | | | | |
| After therapy | P = 0.122 | P = 0.964 | P = 0.7019 | P = 0.5754 | P = 0.7377 |

Observation of the Indexes Before and After Medication

Many indexes such as RBC, WBC, aminotransferase, BUN, Cr are measured before medication. Comparing the two groups with the indexes, most of them have no obvious variance (P>0.05). Though some indexes have evaluation variance, the means are in the normal range (Detail in table 9 and 10). This result suggests that the preparations admitted in the trial and contrast groups are both safe.

6. X² significance of the indexes before and after medication

TABLE 11
X² significance of the indexes before and after medication

| | | degree of | types of | characters of | Induced | papanicolaou |
|---|---|---|---|---|---|---|
| | Massive | cervical erosion | cervical erosion | leucorrhoea | clearance degree | degree |
| Significance and variance | P < 0.05 | P < 0.01 | P < 0.01 | P > 0.05 | P < 0.01 | P < 0.01 |
| test in the trial group | | | | | | |
| before the third therapy | | | | | | |
| (Exact probability) | | | | | | |
| significance variance test | P < 0.05 | P < 0.01 | P < 0.01 | P > 0.01 | P < 0.01 | P < 0.01 |
| in the contrast group | | | | | | |
| before therapy and the | | | | | | |
| third re-examination | | | | | | |
| (exact probability method) | | | | | | |
| Significance variance test | P > 0.05 | P > 0.02 | P > 0.05 | P > 0.05 | P > 0.05 | P > 0.05 |
| of the trial and contrast | | | | | | |
| group before therapy | | | | | | |
| (exact probability method) | | | | | | |
| Significance variance test | P > 0.05 | P > 0.02 | P > 0.05 | P > 0.05 | P > 0.05 | P > 0.05 |
| of the trail and contrast | | | | | | |
| group at the third | | | | | | |
| re-examination | | | | | | |
| (exact probability method) | | | | | | |

In a word, the two preparations both have obvious effects, especially in the trial group for treatment of cervical erosion, which healed rate can reach 88% and total effective rate can reach 100%. They are both able to reduce the vaginal secretion and change its characteristics. At the same time their side effects are also minor, causing no obvious local stimulation and no hurts to normal tissues. For the treatment of cervical erosion, both of them are highly effective, safe and convenient external preparations. Meanwhile as to the reasonable administration manner, cotton ball with tail adheres erosive surface tightly and is convenient to take down by the patient herself, the preparation is accepted by the patients pleasantly.

In conclusion, the preparation (embrocation) provides a new way to cure

1. A preparation for treatment of cervical erosion, wherein comprising products of Nocardia Rubra cell wall skeleton.

2. The preparation of claim 1, wherein comprising pharmaceutically acceptable carriers.

3. The preparation of claim 1, wherein Nocardia Rubra cell is deposited in China General Microbiological Culture Collection Center with deposition No.: CGMCC No0712.

4. The preparation of claim 2, wherein the pharmaceutically acceptable carriers include carbohydrate, glycoside, fat or fatty acid, protein, amino acid, esters, spirits, demasorb, hydroxycarboxylic acid, poly-anion, surfactant, antiseptic, antioxidant and dextran.

5. The preparation claim 2, wherein content of Nocardia Rubra cell wall skeleton is in amount of 0.005 weight %-99 weight % based on the total weight of the preparation.

6. A process and test method of a preparation of treating cervical erosion wherein:
After cultivating, collecting thallus, cell fragmentation, purifying the enzyme, removing the grease, Nocardia Rubra cell wall skeleton is obtained, followed by mixing the Nocardia Rubra cell wall skeleton and the pharmaceutically acceptable carriers to obtain the preparation;
A quality test method includes batch detection of strain of Nocardia Rubra, culture liquid of Nocardia Rubra cell wall skeleton and a final product containing Nocadia Rubra cell wall skeleton.

7. The preparation of claim 6, wherein the pharmaceutically acceptable carriers include carbohydrate, glycoside, fat or fatty acid, protein, amino acid, esters, spirits, demasorb, hydroxycarboxylic acid, poly-anion, surfactant, antiseptic, antioxidant and dextran.

8. The preparation of claim 6, wherein the content of Nocardia Rubra cell wall skeleton is in amount of 0.005 weight %˜99 weight % based on the total weight of the preparation.

9. A method for treatment of cervical erosion, wherein applying pharmaceutically effective amount of Nocardia Rubra cell wall skeleton preparation directly on a suffering site of subject.
